(19) United States
(12) Patent Application Publication
(10) Pub. No.: US 2006/0106374 A1
(43) Pub. Date: May 18, 2006

(54) Expendable optical waveguide with use-tracking feature

(75) Inventor: Kelly Moran, Wilbraham, MA (US)

Correspondence Address: BOLESH J. SKUTNIK, PH. D, JD, CeramOptec Industries, Inc., 515 Shaker Road, East Longmeadow, MA, 01028, US

(73) Assignee: CeramOptec Industries, Inc.

(21) Appl. No.: 11/238,391

(22) Filed: Sep. 29, 2005

Related U.S. Application Data
(60) Provisional application No. 60/628,967, filed on Nov. 18, 2004.

(51) Int. Cl. A61B 18/18

(52) U.S. Cl. 606/16

(57) ABSTRACT

An expendable optical fiber suitable for use in endoscopic medical treatments with a variety of light sources is described. The inventive fiber has means for determining the fiber's cumulative use history and means for enhancing visualization at the treatment site. The means for determining the fiber's cumulative use history and the means for enhancing visualization are located along predetermined portions of the fiber's surface. The inventive fiber provides for optimal color contrast and background for performing medical procedures and allows for the rapid determination of a fiber's cumulative use history by visual inspection.

### DOMESTIC PRIORITY UNDER 35 USC 119(e)

This application claims the benefit of U. S. Provisional Application Ser. No. 60/628,967, filed Nov. 18, 2004, which is incorporated by reference herein.

### BACKGROUND OF THE INVENTION

1. Field of the Invention

The present invention relates generally to medical devices and procedures, and, in particular, relates to means to monitor the use history of optical fibers employed in medical endoscopic treatments, such as lithotripsy.

2. Information Disclosure Statement

Optical fibers are used to deliver light energy in a wide variety of medical applications. Often the area to be treated is located deep within the body and not directly accessible to a light source. As a result, many laser assisted medical treatments and/or surgeries require flexible waveguides to access and deliver light energy to a target site with the body. For medical procedures involving percutaneous entry into the body, optical fiber devices provide a minimally invasive means of treatment and are excellent alternatives to open surgical interventions. For example, procedures such as lithotripsy utilize optical fiber endoscopes to destroy and remove calculi or stones deposited in body lumens such as the bladder. The growing number of commercially available light sources, such as pulsed lasers, diode lasers, and neodymium lasers, emphasize the versatility of optical fiber devices. As a result, optical fibers are utilized in a number of different devices for treatment procedures such as arthroscopic surgeries and laparoscopic surgeries, as well as for diagnostic applications in cardiology and ophthalmology. While the devices used in these procedures may have different names (i.e. endoscopes, fiberscopes, arthroscopes, laparoscopes, etc.) the basic structural elements of the fiber used in these devices are generally the same.

Optical fibers consist generally of an optically transmissive core that is surrounded by a cladding, having a lower refractive index than that of the core. Light propagates along the fiber as a result of the difference between the indices of refraction of the core and cladding materials. In addition, most commercial fibers require a protective jacket which surrounds the outside of the cladding. The jacket is usually a thin layer of metal, plastic or other polymeric material which protects the fiber from scratches and other environmental damage that occurs during shipping, handling, or use. Damage such as scratches and microcracks can compromise the strength and optical properties of the fiber. In some instances, the outer jacket also increases the rigidity of a fiber which is helpful when introducing the fiber into the body. The most common method for adding a protective jacket to an optical fiber involves coating or extrusion of a tubular sheath or sleeve which adheres to the cladding.

Despite the availability of disposable optical fibers, in the medical setting it is often economically advantageous to use an optical fiber a number of times before discarding it. During each use the fiber becomes contaminated and must be cleaned and sterilized before it can be used again. The practice of repeatedly sterilizing and reusing optical fibers, however, can jeopardize patient safety because the structural integrity of the fiber can deteriorate significantly as a result of repeated exposure to sterilization processes. Specifically, most sterilization processes can hasten the growth of flaws which, in turn, deteriorates fiber strength and in vitro performance.

Most surgical instruments are sterilized via autoclaving at high temperatures or by exposure to chemical sterilants. Both types of sterilization procedures can be problematic when used to sterilize optical fibers after a prior use. For example, autoclaving is a widely used sterilization method that subjects optical fibers to high temperatures, moisture, and excessive handling (e.g. bending, flexing, accidental dropping or bumping, etc.). These mechanical and chemical stresses can cause micro-cracking and other degradation damage, which can be exacerbated by the high energy densities carried by the fiber during normal use. Damage incurred by repeated sterilization and reuse is usually undetectable by simple visual inspection of the fiber and can cause the fiber to malfunction and/or fracture in vivo during treatment. Moreover, the tiny glass shards which remain in the body after an in vivo fracture could pose serious health risks for patients.

The potential risks associated with the reiterative sterilization and reuse of optical fibers could be avoided if the “cumulative use history” of a fiber could be readily ascertained before treatment with an optical device. As used herein, “cumulative use history” is defined as the duration of use or approximate number of times an optical fiber has been used in the past. “Multi-use” fibers are fibers that are intended for more than one use, whereas “single-use” or disposable fibers are fibers that are intended to be used once and discarded. As used generally, the distal end refers to the treatment or in vivo end of the fiber, whereas the proximal end refers to the ex vivo end of the fiber. To avoid the potential problems associated with the practice of repeatedly sterilizing and reusing optical fibers, the present invention addresses the need for the inexpensive, reliable, and convenient verification of the cumulative use history of optical fibers.

Several patents disclose methods of marking optical fibers for the purpose of identification. U.S. Pat. No. 4,629,285 by Carter et al. discloses optical glass fibers color coded with a relatively thin coating of colored ultraviolet cured ink.

U.S. Pat. No. 5,277,292 by Bartling et al. discloses an optical fiber having a external color coating with additional different color identification makers in the color coating. The procedure allows for the identification of multi-optical fibers in a communications cable.

U.S. patent application Publication US 2004/0037521 discloses a procedure for applying two coatings to an optical fiber. The outer coating being transparent so the color of the inner coating is visible.

U.S. Pat. No. 5,796,905 by Hoffart et al. discloses optical fibers having color markings. The markings may be bands, or stripes or almost any marking that can be used for identification.

U.S. Pat. No. 6,306,094 by Joseph discloses the placement of bubble generating means on the end of the fibers. When exposed to bodily fluids, bubbles are generated which are visible to ultrasound and thus the location of the needle or catheter may be observed during the procedure.

Therefore, there exits a need for a means for determining the usage on a medical optical fiber.

### OBJECTIVES AND BRIEF SUMMARY OF THE INVENTION

It is an object of the present invention to provide an optical waveguide for the transmission and delivery of light in which the cumulative use history of the waveguide is readily ascertainable by visual inspection.

It is another object of the invention to provide an expendable optical fiber for use in endoscopic applications where a marked portion of the optical fiber enhances visualization at the treatment site during treatment.

It is yet another object of the invention to provide a means for determining the cumulative use history of an optical fiber where a marked portion of the optical fiber also enhances visualization at the treatment site in vivo.

It is still another object of the invention to provide a method of recording and determining the cumulative use history of an optical fiber irrespective of fiber geometry, composition or configuration, and includes both multi-use and single-use optical fibers.

Briefly stated, the present invention provides an expendable optical fiber suitable for use in medical procedures with a variety of light sources. The inventive fiber has an outermost surface having predetermined portions with means for enhancing visualization of the treatment area and means for rapid and convenient determination of the fiber's cumulative use history by visual inspection. The means for enhancing visualization provides optimal color contrast during medical procedures, such as lithotripsy or tissue ablation. In one embodiment, the means for visual enhancement and the means for determining cumulative use history are located along the same predetermined portion of the fiber. In another embodiment, the means for visual enhancement and the means for determining use history are located along different predetermined portions of the fiber surface.

The above and other objects, features and advantages of the present invention described herein will become apparent from the following description read in conjunction with the accompanying drawings. BRIEF DESCRIPTION OF FIGURES

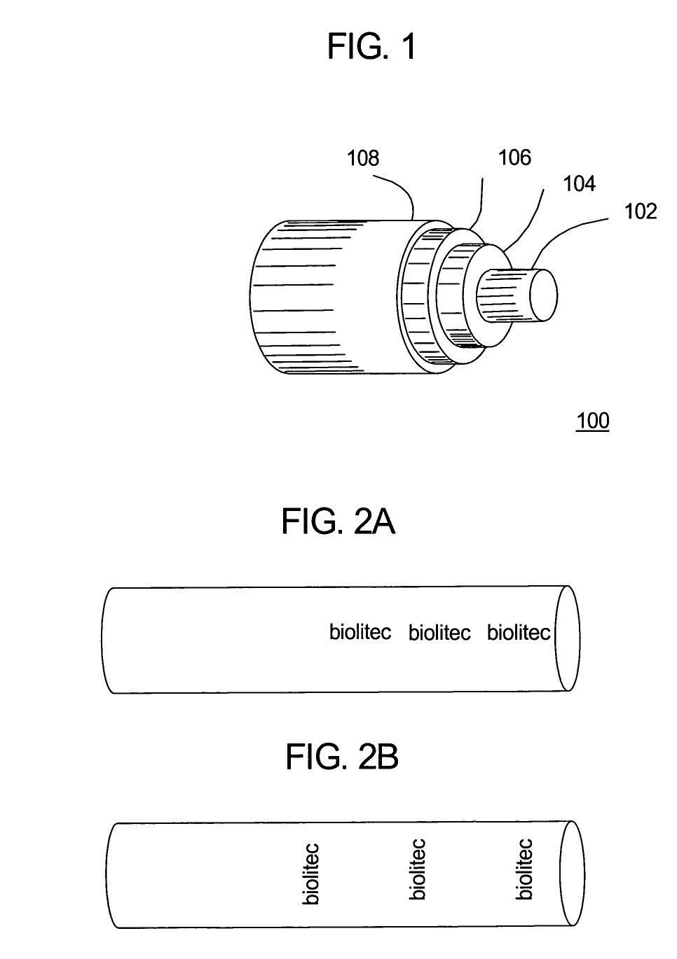

FIG. 1 illustrates by perspective view an optical fiber of the present invention.

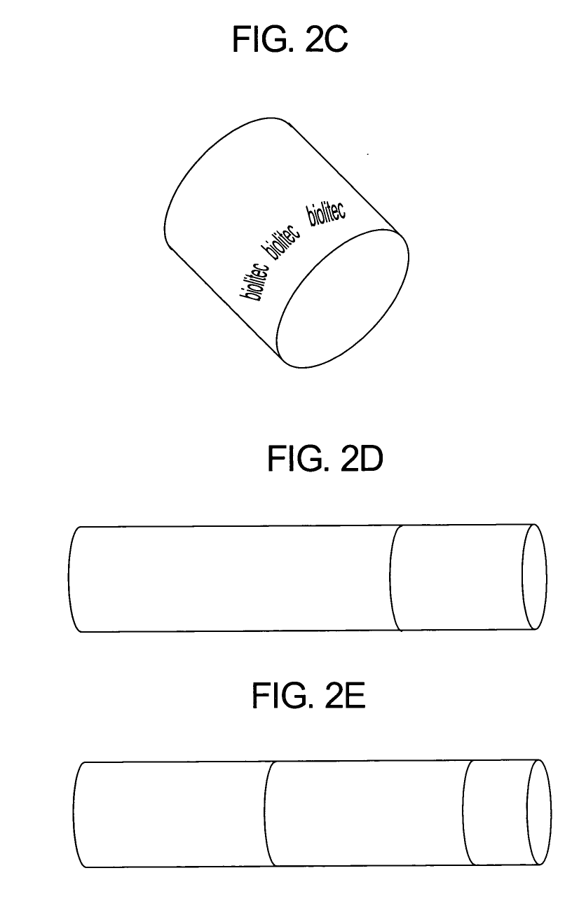

FIG. 2A to 2H illustrate examples of markings placed on the optical fibers in accord with the present invention.

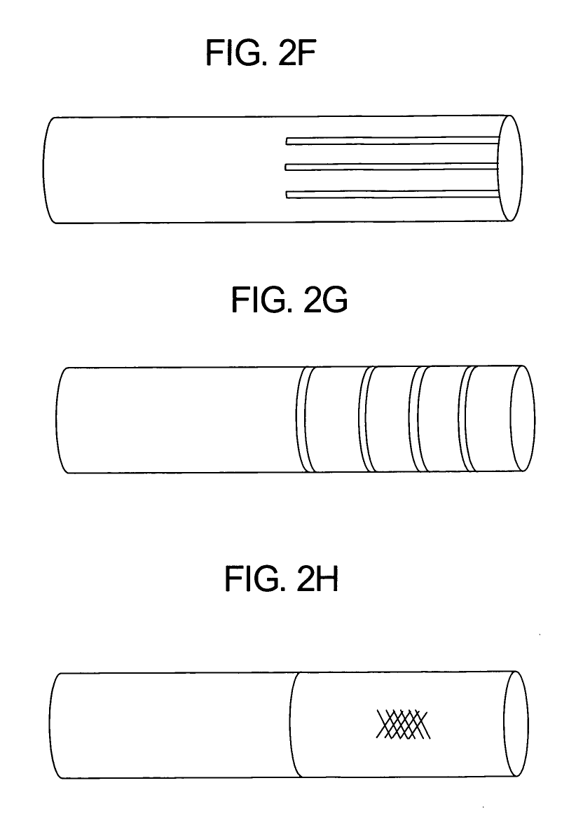

FIG. 3 illustrates one example of an optical fiber configuration contemplated by the present invention having a colored section on the in vivo end of the optical fiber for enhanced visualization within the body.

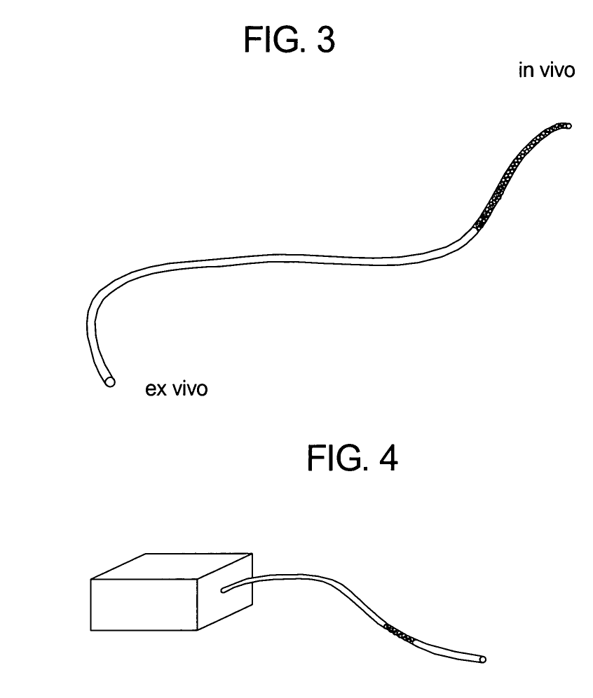

FIG. 4 illustrates another example of a marking intermediate on an optical fiber having a configuration in accord with the present invention.

### DETAILED DESCRIPTION OF THE PREFERRED EMBODIMENTS

The present invention provides an economical, expendable optical fiber assembly for use in endoscopic procedures in which the cumulative use history of the optical fiber can be determined by simple inspection.

FIG. 1 illustrates an optical fiber 100 in accord with the present invention. Optical fiber 100 includes a core 102 which is concentrically located within a cladding layer 104 and may also include a buffer layer 106. An outermost surface 1 10 on optical fiber 100 is provided by a jacket layer 108.

In a preferred embodiment, a predetermined length of the optical fiber jacket is marked using a colored portion. The marking is placed on the outermost surface of the fiber during the manufacturing process or post-production in a variety of ways known in the art, but markings may be placed on or in the cladding or buffer layer. Introducing the marking to the cladding or buffer layer can be achieved by any of the methods described above or, preferably, by the use of an inner sleeve which is visually distinguishable from the outer jacket layer.

The present invention is not limited by the means used for introducing the marking onto a particular surface of the fiber, but the marking may be introduced by shrink wrapping a colored portion of a predetermined length onto the outermost surface of the fiber. Other techniques of placing a colored portion on the outer surface include using ink. Alternatively, the fiber may be marked using silicones, polymers, paints, or laser etching, stamping, dipping, spraying, templates, or carrier-assisted deposition, a texture or a finish (e.g. matte vs. high gloss; smooth vs. rough). Further, the markings may consist of a material that interacts with the laser radiation or of a material that emits bubbles when exposed to internal bodily fluids as a means for tracking the cumulative use history. For example, if the material is exposed and changes color that optical fiber may not be reused until that colored section is removed. This material may be placed on a marked portion that is either non-continuous or continuous. It is further possible to have a marked portion that enhances visualization and another marked portion that shows the cumulative use history or they could be combined. For example, the marked portion may change color for increase visualization but also indicate usage. For example, an optical fiber may have four colored bands indicating that the optical fiber can be used only four times. After each use, one of the bands is removed. Further, each band may have a unique color when it is exposed to bodily fluids, temperature, etc. One band may be blue, the next green, the next yellow and the last red. Further, this identification may be carried out by materials that are visible under ultrasound, x-ray, or exposed to laser radiation of a particular wavelength.

The present invention also contemplates the use of jackets that may develop a marking after exposure to heat, to a chemical, or to a photochemical process.

Moreover, the marking of the present invention is not limited to a colored mark. For fibers having a colored jacket or colored outermost surface, the absence of color along a portion of the fiber is also considered a marking. Further the marking may emit light when exposed to a particular wavelength thus including a florescent or phosphorescent marking along a predetermined portion of the fiber that is visible only under certain wavelengths of light (such as UV).

FIG. 1 illustrates a preferred embodiment of the present invention where a marked portion 1 10 of the fiber is a continuous band of a predetermined length on the outermost surface or jacket of the fiber 100. See FIG. 3 also. The present invention is not limited to marking a portion of the fiber in a continuous band, but a marked portion may also be referred to as a “non-continuous” marking. Examples of non-continuous markings may be a single or multiple stripes of a predetermined length on the outermost surface of the fiber as seen in FIG. 2F.

Other examples of non-continuous markings are shown in FIGS. 2A to 2H and may include, but not limited to, stripes, dots, dashed lines, crosshatches, etc. Or the optical fiber is marked with the name of a company, the product name, insignia, logo, trademark, part or product number, or other product identification means (such as patent application number(s) or “patent pending”) in a staggered, circular or linear fashion. In an exemplary embodiment, the optical fiber of the present invention is marked with the name “biolitec.” See FIGS. 2A to 2C. The non-continuous markings of the present invention are not required along the entirety of the predetermined portion of the fiber nor does the marking need to completely cover or encircle the optical fiber along the predetermined length of the outermost surface.

Alternatively, the outermost surface of the fiber can be marked using an additional layer of a very thin material that is affixed to or embedded in the outermost surface using heat, epoxy or other chemical or mechanical means. This additional layer can be colored, patterned, or textured or possess any combination of the marking embodiments as described herein.

As with the other embodiments of marking described above, this additional layer would not cover the entire length of the fiber but would be limited to a predetermined length along or near the distal (in vivo) end 302 or towards the proximal (ex vivo) end 300 of the fiber.

In a preferred embodiment, the marked portion of the fiber also provides for optimal color contrast or background for enhancing visualization at the treatment site during medical procedures. In an alternate embodiment, the marked portion has no effect on visualization at the treatment site.

Accordingly, the present invention is not limited to the end of the fiber that bears the marking. In one preferred embodiment, shown in FIG. 3, the marking is located along a predetermined length of the distal or treatment (in vivo) end 302 of the fiber. In another preferred embodiment, the marking is located along a predetermined length of the ex vivo end 400 of the fiber and remains visible during treatment of the patient as shown in FIG. 4.

Further, the optical fiber may contain markings of different types wherein information of a particular nature is displayed by each type of marking.

It is thus a critical feature of the present invention that the marked portion wherever located serves as a visual record of the fiber's “cumulative use history” because a discrete section of the marked portion is removed or stripped prior to sterilization, or abraded during use. A fiber's cumulative use history can be determined if the marked portion does not remain ex vivo and/or visible during a medical procedure. As such, any reduction in the predetermined length of the marked portion or the inability to view the marking during use of the fiber will serve as a visual record for determining a fiber's cumulative use history. The present invention is not limited by the means for reducing the length of the marked portion or for the loss of visibility of a marking during a medical procedure.

Commercial fibers are manufactured and sold as either reusable, i.e. “multi-use,” fibers, or disposable, i.e. “single-use,” fibers. Unlike single use fibers, multi-use fibers are used again after the distal end is reconditioned and the entire fiber is sterilized. In one embodiment of the invention, a discrete section of the marked portion is removed by stripping off the outermost surface of the fiber. Where the marked portion is located along the distal or treatment end of the fiber, it is typical to remove approximately 1-2 cm of the marked portion prior to reusing the fiber. After stripping, the bare fiber is cleaved using a cleave tool.

However, the length of the predetermined marked portion can be reduced by in vivo abrasion or erosion that occurs at the fiber tip during use. Specifically, the fiber tip can break apart when the high energy laser beam is released from the fiber in short pulses. Thus, it is preferred that an optical fiber of the present invention have a predetermined marked portion of at least 1 to 50 cm in length and that at least 1-2 cm of the predetermined marked portion of the fiber is removed in preparation for reuse. In an exemplary embodiment, an optical fiber having a marked portion of a predetermined length that measures between 25 μm to 2 cm at its distal end is defined as a single-use or disposable fiber. For the single-use embodiment, the cumulative use history of the fiber is evidenced by any reduction in the length of the marked portion or by any removal of the marked portion, whether the reduction/removal is caused by in vivo or ex vivo abrasion or erosion or removal of the marking.

The marked optical fibers of the present invention can be used in combination with diffuser tips, shaped tips, needles, catheters, or any other devices that aid in light delivery to body lumens or interstitial sites known in the art.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the figures and appended claims.

What is claimed is:

1. An optical waveguide for the transmission and delivery of light energy from a source of said light energy comprising:
means for determining a cumulative use history of said optical waveguide, said means being located on said optical fiber and being readily ascertainable by visual inspection.

2. An expendable optical fiber comprising:
at least one core;
at least one cladding layer over said core;
a jacket layer over said cladding layer, said jacket layer having an outermost surface, said outermost surface having thereon said means for determining the cumulative use history of said optical fiber.

3. An optical fiber according to claim 2 wherein said outermost surface of said optical fiber includes means for enhancing visualization or color contrast during medical procedures along a distal end of said optical fiber.

4. An optical fiber according to claim 2 wherein said means for determining said cumulative use history is a marked portion of a predetermined length on said outermost surface.

5. An optical fiber according to claim 4, wherein said marked portion is comprised of a continuous or non-continuous marking.

6. An optical fiber according to claim 5, wherein said non-continuous marking is selected for the group consisting of stripes, dots, dashed lines, cross-hatch, company name, manufacturer name, insignia, logo, trademark, part number, product number, and other product identification means.

7. An optical fiber according to claim 6, wherein said other product identification means at least includes lot numbers, serial numbers, patent or patent application numbers, or the phrase “patent pending.”

8. An optical fiber according to claim 5, wherein said continuous marking is comprised of a band of at least one color.

9. An optical fiber according to claim 5, wherein said continuous marking is comprised of a band that is without color.

10. An optical fiber according to claim 4 wherein a discrete section of said marked portion must be removed prior to reuse of said optical fiber.

11. An optical fiber according to claim 10 wherein said cumulative use history is readily ascertainable by visual inspection.

12. An optical fiber according to claim 11 wherein said predetermined length of said marked portion provides a visual record of how many times said optical fiber has been used.

13. An optical fiber according to claim 12 wherein said predetermined length is between 25 μm and 50 cm in length.

14. An optical waveguide according to claim 1 wherein said cumulative use history is determined by a marked portion of a predetermined length on said outermost surface.

15. A single-use optical waveguide comprising a marked portion of a predetermined length on the outermost surface at its distal end, wherein said marked portion is at least between 25 μm to 2 cm in length.
